(19) United States
(12) Patent Application Publication
(10) Pub. No.: US 2023/0011670 A1
(43) Pub. Date: Jan. 12, 2023

(54) RAPID EXCHANGE ENDOSCOPE SYSTEM

(71) Applicant: The Regents of the University of California, Oakland, CA (US)

(72) Inventors: Matthew Kohanfars, Encino, CA (US); Farshad Ahadian, Rancho Santa Fe, CA (US); Yu Li, La Jolla, CA (US); Karcher Morris, Huntington Beach, CA (US); Frank Talke, Rancho Santa Fe, CA (US)

(21) Appl. No.: 17/782,443

(22) PCT Filed: Dec. 17, 2020
(86) PCT No.: PCT/US2020/065674

§ 371 Date: Jun. 3, 2022

Related U.S. Application Data
(60) Provisional application No. 62/949,876, filed on Dec. 18, 2019.

(51) Int. Cl. A61B 1/00; A61B 1/005; A61B 1/267

(52) U.S. Cl. CPC A61B 1/00105; A61B 1/0057; A61B 1/00128; A61B 1/00119; A61B 1/267; A61B 1/0052; A61B 1/00147; A61B 1/00135

(57) ABSTRACT

A rapid exchange detachable endoscope includes a transmission system having a shaft with two linear translation nuts mounted on oppositely threaded portions of the shaft so that rotation of the shaft in one direction moves the two linear translation nuts further away from one another and rotation of the shaft in an opposite direction moves the two linear translation nuts closer together to control an insertion tube tip. A control handle can be attached and detached from the transmission system, allowing an insertion tube associated with the transmission system to remain in vivo and allow for exchange of overtubes.

### PRIORITY CLAIM AND REFERENCE TO RELATED APPLICATION

The application claims priority under 35 U.S.C. § 119 and all applicable statutes from prior U.S. provisional application Ser. No. 62/949,876, which was filed Dec. 18, 2019.

### FIELD

A field of the invention is endoscopes, including bronchoscopes.

### BACKGROUND

Endoscopes are now widely used for examination procedures and surgical procedures. Endoscopes are expensive precision instruments consisting of a handle with control features, and an elongated tube or tubes that carry instruments, such as cameras and surgical instruments through a small body lumen to a point of interest for examination or surgery. The elongated tube is dimensioned to fit within and carry an endotracheal tube.

Artisans have recognized the potential benefit of having a handle that is detachable from the elongated tube or tubes. One example is described in Krupa et al., U.S. Published Patent Application 20080214896. The described handle includes two bulky pieces that are joined via a complex alignment and latching procedure. Gears must be aligned and set and then a latch closed. Detachment also requires multiple steps with latches and keys and pulling of one of a bulky “elongation portion from a handle”. The elongation portion has the elongated shaft integrated with it and remains attached to the shaft when separated from the handle. In addition, when coupling the connection must be made to interface the optics of the device. The light source must be properly aligned for illumination and visualization through an optical lens from the proximal location of the control handle. If not properly aligned, image output will be distorted, and a coherent image will not be displayed.

Lowery et al, U.S. Pat. No. 4,624,243 describes a detachable viewing device having a reusable eyepiece. The viewing device can be used with an endoscope with a reusable eyepiece and a disposable distal section. The eyepiece requires a predetermined angular orientation with respect to the distal section to function. The connection between the eyepiece and the distal section is suitable for exchanging a distal section when the instrument is not in use, while the eyepiece and distal section are configured to remain together during a procedure. The device does not allow the distal end to be operated by a user and does not have ports for suction and oxygenation. The device also does not allow for maneuverability of the distal end to be operated by a user fails to provide ports for suction and oxygenation support. Marini U.S. Pat. No. 6,077,290 describes an endoscope with a removable control handle. A ball and socket connection is made to the tubular member, and is locked by a threaded locking knob. The handle includes a pivoting, scissors-style operation. The tubular member and handle are uniquely aligned via a slot. Attaching and detaching the handle to the tubular member is relatively complex due to the slot and threaded locking knob. When connected the scissor-style handle allows for manipulation of the forceps located at the distal end of the device.

Tip control is necessary for modern endoscopes but is difficult to implement with a detachable handle. One solution is proposed in Hunter et al., US Published Application 2015/0011830 and in Chen et. al, “Disposable Endoscope Tip Actuation Design and Robotic Platform,” 32 Annual International Conference of the IEEE EMBS, Buenos Aires, Argentina, Aug. 31-Sep. 4, 2010. The described design includes a rotary motor around which are oppositely wound ends of two opposing cables that extend to the distal tip. A motor located at the distal end of the device rotates the shaft to bend the tip and a reverse rotation bends the tip in a different direction. The drive shaft, typically called a capstan drive, relies on the winding about a shaft having two strings that are wrapped in opposite directions on the shaft. Due to the mechanical disconnect created by the capstan drive and the distal motor, the operator lacks mechanical feedback from the distal end of the elongated shaft. Such tactile feedback is important to safe insertion into patients' airways. Capstan drives also suffer from high stress being placed on the control wires connected to the endoscopes distal linkage system. The tightly wound wires around the small diameter shaft quickly become frail due to the repetitive load.

### SUMMARY OF THE INVENTION

Preferred embodiments include a rapid exchange detachable endoscope including a transmission system having a shaft with two linear translation nuts mounted on oppositely threaded portions of the shaft so that rotation of the shaft in one direction moves the two linear translation nuts further away from one another and rotation of the shaft in an opposite direction moves the two linear translation nuts closer together to control an insertion tube tip. A control handle can be attached and detached from the transmission system, allowing an insertion tube associated with the transmission system to remain in vivo and allow for exchange of overtubes.

### BRIEF DESCRIPTION OF THE DRAWINGS

FIGS. 1A-1B show a preferred endoscope including a control handle and transmission system of the invention with the transmission respectively attached and released;

FIGS. 2A-2C are views of the preferred control handle shown in FIGS. 1A-1B;

FIGS. 3A-3B are views of the preferred transmission system of FIGS. 1A-1B;

FIGS. 4A-4C are views of the preferred detachment mechanism of FIGS. 1A-1B;

FIGS. 5A-5D are view of another control handle and transmission system of the invention.

### DETAILED DESCRIPTION OF THE PREFERRED EMBODIMENTS

A preferred embodiment of the invention is a rapid-exchange detachable endoscope assembly that contains a control body and separable insertion tube that form a complete endoscope. The endoscope assembly includes an embedded transmission system. An endotracheal tube can be easily exchanged after insertion of the endotracheal tube carried by the insertion tube due to the decoupling of the control handle from the insertion tube. In other words, an endotracheal tube can be replaced with a different endotracheal tube after removal of the control handle by sliding the first endotracheal tube out and inserting the second endotracheal tube in the patient over the already positioned insertion tube Channels, electrical connections and a transmission system in the insertion tube can be quickly and simply detached, preferably by the press or twist of a button. When connected to the handle, operation of sensors in a proximal portion of the insertion tube and mechanical movements of a distal end of the insertion tube can be conducted from the control handle. The control knob located on the handle enables operation of the transmission system in the insertion tube. The system, preferably a threaded design, allows for distal tip articulation. Preferably, there are at least two threads that independently control length and angulation of the distal tip of the insertion tube.

A preferred detachable coupling between the control handle and the insertion tube provides for separation of the control handle from the insertion tube of the endoscope in order to allow for safe exchange of endotracheal tubes during intubation of patients in critically ill conditions, followed by a change of the endotracheal tube and reattachment of the control handle and direct visualization of the airway for confirmation of proper tube placement. Endoscopic visualization of the airway is the gold standard for confirmation of proper placement of the endotracheal tube under many of these critical conditions. The preferred endoscope assembly serves both functions of endotracheal tube exchange as well as endoscopic visualization.

Additional benefits of the preferred detachable endoscope assembly include a uniform diameter of the insertion tube along its entire length. This is important for unimpeded passage of endotracheal tubes over the insertion tube of the endoscope. Preferred embodiments include a device that is a disposable endoscope insertion tube combined with a permanent handle. Preferred designs provide the ability to scale the transmission system, which allows larger channel optimization. Detachment is a single step process, allowing a quick connection/disconnection. In preferred embodiments, the control handle can be attached independent of distal-end angulation. The preferred interface and detachment features do not require a significant upward force to attach/detach.

The device can include additional features, such as a temperature probe, pressure/force sensor, or x-ray detectable insert desired at contact edge or along the insertion tube for feedback to the user. By using a radio opaque material at the tip, a practitioner can use tools to determine insertion depth.

Preferred devices are simple to use, even for less experienced practitioners. A preferred control handle houses a male connector that engages into a port of the insertion tube at the proximal end of the insertion tube. This connection allows direct manipulation of the translational movement of the transmission system. As the translational movement is driven by the operator, the end-effector rotates in response to this change in position on the transmission system. The transmission system is designed with two types of threads, a left-handed thread and right-handed thread separated by a spacer. Two precisely designed, low friction threaded nuts translate along these threads. The threaded nuts are connected to wires attached to the end effector through the insertion tube. When the transmission system is rotated, the mechanism behaves similarly to a pulley system that changes the length of the wires resulting in the angulation of the distal end. In addition, when the handle needs to be disconnected from the insertion tube, a push-button on the control handle can be actuated to disengage the two parts.

Preferred embodiments of the invention will now be discussed with respect to the drawings and with respect to experimental devices. The drawings may include schematic representations, which will be understood by artisans in view of the general knowledge in the art and the description that follows. Features may be exaggerated in the drawings for emphasis, and features may not be to scale.

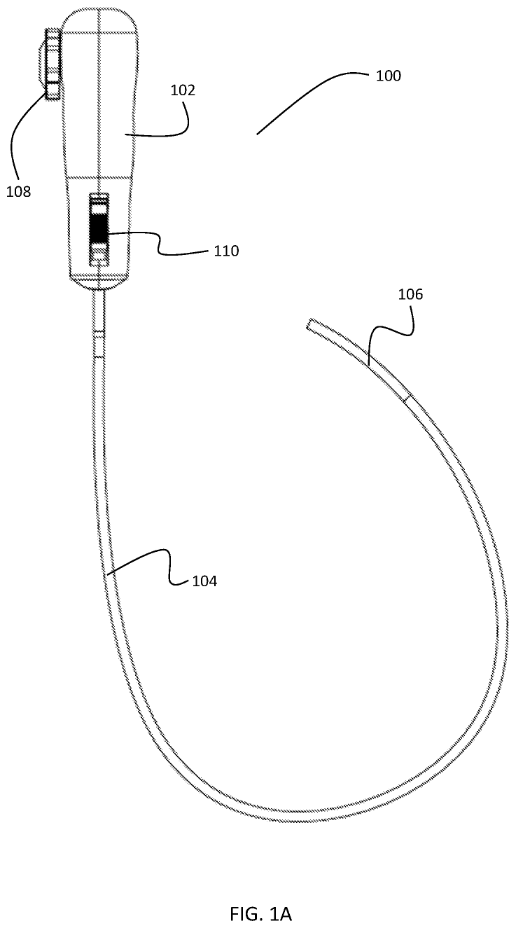

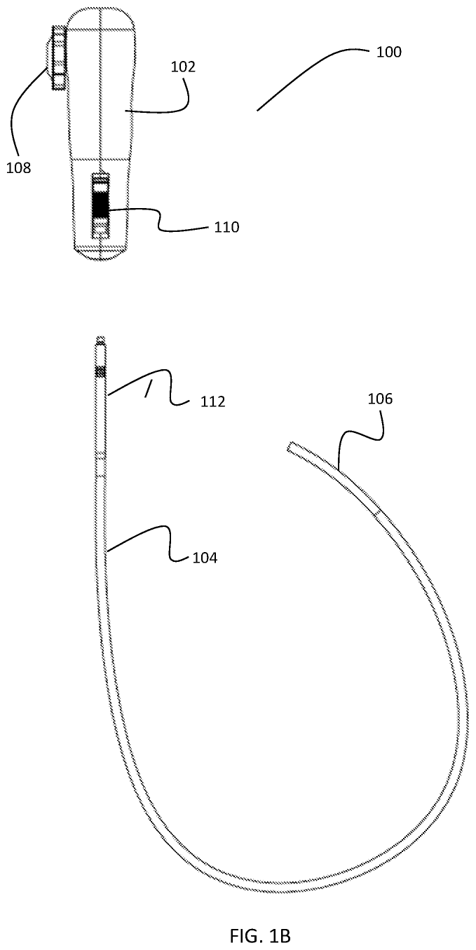

FIG. 1A shows a preferred embodiment rapid exchange detachable endoscope assembly 100. The endoscope assembly 100 includes a control handle 102 removably attached to an insertion tube 104. The insertion tube has a length and diameter selected to enter a body lumen to a point of interest within the body lumen. A tip 106 of the insertion tube 104 is controlled by a rotatable control knob 108 on the control handle 102. The control handle 102 is preferably sized and contoured to fit the shape of a user's hand, while allowing the user to actuate a release switch or button 110 with the same hand. FIG. 1B shows the endoscope 100 with the control handle 102 detached from the insertion tube 104. The insertion tube 104 is connected to a transmission system 112 that interacts with a gearbox (not shown in FIG. 1B) inside the control handle 102. The insertion tube is sized to carry an endotracheal tube (not shown) and allow in vivo exchange of endotracheal tubes.

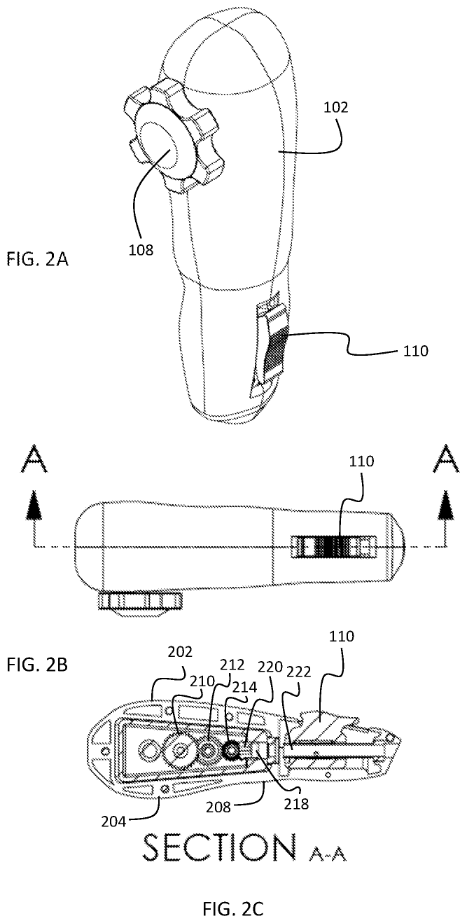

FIGS. 2A-2C are more detailed views of the control handle 102. The handle includes contours 202, 204 and 208 that provide a comfortable grip and allow actuation of the button 110 to quickly detach the control handle 102. The control knob 108 rotates a main gear 210 which transmits rotation to second gear 212 and third gears 214 having axes of rotation that are parallel to the main gear 210. Rotation of the third gear 214 is meshed with the bevel gear 220, changing the axis of rotation to be parallel with the connector 218. This shift in the axis of rotation aligns the motion of connector 218 to the transmission system 112. A tube 222 is part of an assembly to attach and detach the transmission system 112 to the control handle 102.

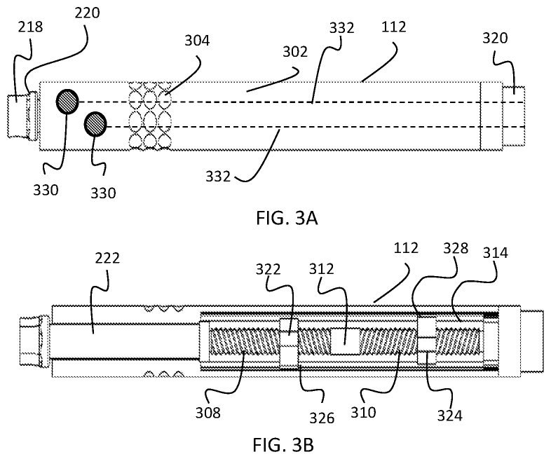

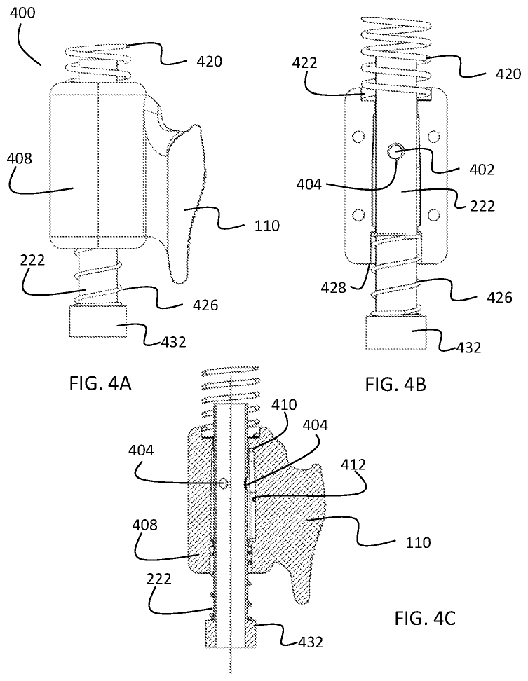

The transmission system 112 is shown in FIGS. 3A-3B. It includes an outer housing 302 having a plurality of detents or hollows 304 to interact with ball bearing attachment in the control handle 102. Providing the hollows in a pattern that surrounds the outer housing allows the transmission system to interact with the detachment system of FIGS. 4A-4C in any random orientation. The outer housing 302 is generally elongate and cylindrical, and approximately the diameter or slightly larger than the endoscope insertion tube 104. The transmission system 112 includes two oppositely threaded portions 308 and 310 separated by a middle portion 312 within a chamber 314 of the housing 302. A tube connector 320 is sized to mate with the insertion tube 104. The chamber 314 is shaped to guide linear translation nuts 322 and 324 that move in opposite directions because of the opposite threaded portions 308 and 310 when the shaft 218 is rotated in response to the gears 210, 212 and 214 as controlled by the control knob 108. The linear translation nuts 322 and 324 are respectively connected to separate control wires 326 and 328 that control the tip 106 of the insertion tube 104. During operation, wires 326 and 328 will move in the opposite direction with a ratio of 1-to-1. When offset from its neutral position, where both 322 and 324 are at the center of their threaded section 308 and 310 respectively, only 1 wire will be in tension. Guided nut 322 and 324 should be made out of compatible materials for low friction or lubricated. The outer housing 302 can also include one or more electrical contacts 330 to interact with corresponding contacts (not shown) on the control handle 102 or another operational handle configured to attach and detach from the transmission system 112 in the same manner as the control handle. The electrical contacts 330 provide signals from sensors and devices in the insertion tube 104 via embedded electrical leads 332 that extend within the outer hosing 302 and the insertion tube 104. All conventional sensors and optics for endoscopy can be accommodated. The transmission system 112 is shown separately for purposes of illustration, while it is permanently and integrally attached, such as by adhesive or welding, with the insertion tube 104 via the tube connector 320, through which the embedded electrical leads extend into the insertion tube 104 and sensors included therein.

FIGS. 4A-4C show details concerning the button 110 and an associated detachment assembly 400 that interact to hold and release the transmission system 112. The detachment assembly operates as a ball bearing engaging system. Preferably, three ball bearings 402 (one shown in FIG. 4B) are angularly separated, e.g. by 120 degrees) to interact with holes 404 in on tubing 222 to allow engagement with the detents or hollows 304 on the outer housing 302 of the transmission system 112. The ball bearings 402 are held in place by a button housing 408. An inner profile 410 of an inner cavity 412 is chamfered to operate as a cam for the ball bearings 402. At a resting position, the button housing is pushed down by an upper spring 420 that is seated in a seat 422, in which position the ball bearing align with the holes 404 to allow engagement with the detents or hollows 304 if the transmission 112 is inserted to attach the insertion tube 104. A lower spring 426 seated in a seat 428 is less stiff than the upper spring 420. The lower spring 426 helps position the tube 222 by pushing against its collar 432. To detach the insertion tube 104, the button 110 is pushed up by the user so that spring 420 will be compressed and spring 426 will be at rest, which moves the ball bearing 402 out of the holes 404 and out of engagement with the detents or hollows 304.

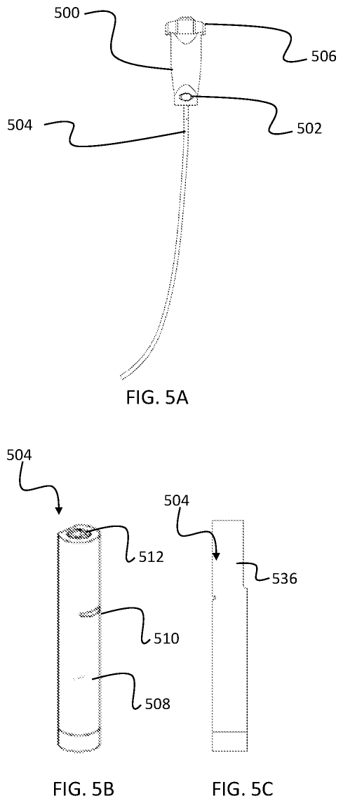

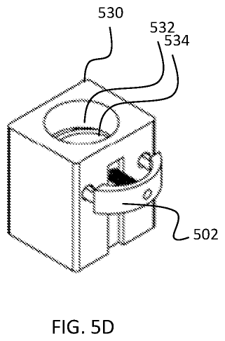

FIG. 5A shows another preferred control handle 500 with a release button 502 attached to a transmission system 504 at the top of an insertion tube (not shown). The transmission system 504 functions as the one shown in FIG. 3B and is also illustrated in FIG. 5B. A rotatable control handle 506 is used to operate the transmission system. FIGS. 5B-5C shows the transmission system 504, with an outer housing 508 including a slot 510 that interacts with a detachment system that includes the release button 502. An adaptive connection to the gearbox housed in 500 (see FIGS. 3A-3B) located at 512 rotates the shaft 218 to create the linear action with the linear translation bolts (see FIG. 3B). FIG. 5D shows a detachment system 530 that holds the transmission system via spring force within a lumen 532 of the detachment system. The detachment system 530 includes a tongue or member 534 that engages slot 510 under spring force and removes the ability of upward and downward movement of 504. To disengage the system, button 502 must be pushed inwards, releasing the tongue or member 534 from the slot 510 and thereby releasing the transmission system 504 from the detachment system 530. A shaped portion 536 of the transmission's housing serves as an alignment key with the lumen 532, to orient the transmission system 504 such that slot 510 and tongue or member 534 align. The tongue or member 534 will automatically insert into the slot 510 upon aligned insertion. Then, button 502 must be pressed to retract the tongue or member 534 to release the transmission 504.

Example preferred medical applications of the invention include bronchoscopy, colonoscopy, endoscopy, esophagoscopy, fiber-optic bronchoscopy, indirect laryngoscopy, intubation, laryngoscopy, nasal endoscopy, panendoscopy, pediatric endoscopy, rhinoscopy, sigmoidoscopy, transnasal esophagoscopy, and upper endoscopy

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims.

1. A rapid exchange endoscope assembly, comprising:
a transmission system having a shaft with two linear translation nuts mounted on oppositely threaded portions of the shaft so that rotation of the shaft in one direction moves the two linear translation nuts further away from one another and rotation of the shaft in an opposite direction moves the two linear translation nuts closer together;
a first endoscopic tip control wire attached to one of the two linear translation nuts;
a second endoscopic tip control wire attached the second of the two linear translation nuts;
a control handle with a detachment mechanism, the detachment mechanism accepting and locking the transmission system into an attached position and releasing the transmission system from the control handle via a button;
an actuator associated with the control handle having a rotation mechanism to rotate the shaft of the transmission system when the transmission system is in the attached position.

2. The system of claim 1, wherein the detachment mechanism and the transmission system are configured such that the transmission system can be inserted into the attached position independent of orientation.

3. The system of claim 1, wherein the transmission system comprises:
an outer housing having a plurality of detents located to interact with one or more ball bearings of the detachment mechanism; and a chamber configured to guide the two linear translation nuts.

4. The system of claim 3, wherein the transmission system comprises a drive connector that extends out from one end of the outer housing that interacts with the rotation mechanism.

5. The system of claim 4, wherein the transmission system comprises a tube connector on an opposite end of the outer housing.

6. The system of claim 3, wherein the outer housing is elongate and cylindrical and approximately the diameter or slightly larger an endoscope insertion tube.

7. The system of claim 1, wherein the detachment mechanism comprises: a tube that accepts an outer housing of the transmission system, the tube being within a button housing that includes a button;
at least one ball bearing held by the button housing, sized to interact with at least one hole in the tube;
upper and lower springs biasing the tube with respect to the button housing in a position that aligns the at least one ball bearing into the at least one hole, wherein the button is configured to accept upward force to compress the upper spring and move the at least one ball bearing out of the at least one hole.

8. The system of claim 8, wherein the at least one ball bearing comprises a plurality of ball bearings arranged at a plurality of angular positions around the button housing and the at least one hole comprises a plurality of holes and at the plurality of angular positions.

9. The system of claim 7, wherein the outer housing of the transmission system comprises a pattern of detents or hollows to engage at least one ball bearing.

10. The system of claim 9, wherein the pattern of detents surrounds the outer housing of the transmission system.

11. The system of claim 9, wherein the outer housing comprises electrical contacts connected to electrical leads embedded in the outer housing and extending into an insertion tube connected to the transmission system.

12. The system of claim 1, comprising an insertion tube extending from the transmission system, an endoscopic tip of the insertion tube being connected to the first and second endoscopic tip control wires.

13. The system of claim 12, wherein the insertion tube is configured to fit into and carry an endotracheal tube.

14. The system of claim 13, wherein the insertion tube is configured to allow exchange of endotracheal tubes in vivo when the control handle is detached.

15. The system of claim 1, comprising ball bearing to detent connections between the transmission and the detachment system that provide multi-orientation insertion and default to a secured locked position.

16. The system of claim 1, comprising a diameter in the insertion tube and the outer housing of the transmission that allows for rapid removal of overtubes through the detachment end.
